(19) United States
(12) Patent Application Publication
(10) Pub. No.: US 2018/0243346 A1
(43) Pub. Date: Aug. 30, 2018

(54) Discovery of Root Cause of Autoimmune Disease; Rheumatoid Arthritis and Osteoarthritis

(71) Applicant: Majid Kazemi, Smithtown, NY (US)

(72) Inventor: Majid Kazemi, Smithtown, NY (US)

(21) Appl. No.: 14/488,576

(22) Filed: Sep. 17, 2014

(51) Int. Cl. A61K 35/74

(52) U.S. Cl. CPC A61K 35/74

(57) ABSTRACT

The root causes of autoimmune diseases are deformed human good bacteria that are done by chemical, environmental, X-Rays, microwave, UAV agents.

The pathway to cancer is from start off with autoimmune disease then after growing and combining with other microbes become cancerous cell. The microbe enters human body over a period of time which it could be one time entry or many times and it does not distinguish the infected host. Is it strong or weak? Is it old or young? Is it male or female? The source of human Autoimmune Disease specially Arthritis has not been known to medical professional field until now through this research, since the nature of disease that slowly enter to human body and stays there for long period in some and short in some other, then after accumulation of microbes population in body to a threshold, human body starts to feel the pain of this disease which it causes discomfort in joints and muscle. The deformed DNA bacteria which it is done by deforming agents are the cause of the disease. Most researchers are trying to find cure for the disease without knowing what is the sources, how the disease are created at the first place? This is about research of how AID (Auto Immune Disease), arthritis microbes enter the body, how does it stay there undiscovered by human Autoimmune System? How does it propagate? How does it reshape itself and fool autoimmune system? Some of these are matters of investigation through this medical disease research. Unknowingly, human put the deformed DNA bacteria in their body through food system especially dairy product and yeast. They enter into human digestion system, to blood stream and to neuron, muscle then to joints. As it enters to digestion system in the gut, the deformed DNA bacterium injects its deformed DNA sequences bacteria in the DNA sequences of human good bacteria and it makes transformation. When deformed DNA bacteria dose mutate human DNA, body immune system does not recognize it as a major change DNA at beginning since few nucleotide changes of deformed DNA nucleotide base pairing such as A, C, G, T, and U are not recognized by autoimmune system and it enter the blood stream. Some of the same deformed good bacteria enter blood stream without being recognized by autoimmune system. Then, the microbe looks for soft tissue for habitat which it is at joints and resides there until an injury happen or until the population of microbe increases to a threshold level at joints and some by DNA replication and re-entry of more deformed bacteria with the same structure. Then, it starts fight with body immune system which it causes inflammation and pain. There are few procedures that can be applied to AID or arthritis, since the cause is bacteria so it has to be eliminated by increasing the population of the same good bacteria of the same DNA structure which it should be used a specific probiotic that has the same DNA structure of human bacteria that had been deformed. Also, some medical professional use specific antibiotic to kill off the complexes of deformed one. Then by use of probiotics treatment and prevention of entry of any more deformed bacteria in to body you can control the disease. Then, mutation that happened to human DNA must be repaired by giving probiotics of the specific kind good bacteria that infected the joints and by gene therapy.

How does Rheumatoid Arthritis form in patient body? We have good copies of every bacterium of nature or anti-foreign bacteria in our gut, intestine, exit and entrance of human body in form of so called antibody agent that is also exist in nature. We have billions of good strains of varieties of good bacteria or anti-bacterial in our body, gut, mouth, noise, ear, and intestine that exist in outside of human body which it does attack the entry of small amount foreign bacteria or harmful bacteria in body. The following agents change the genomic structure of bacteria genes in human when they become deformed by heating, radiation material such as microwave oven x-Ray, dry freeze, by acid chemical, indirect sun light, long time exposure to room temperature, strong acidic food with low PH, heavy metals such as mercury, lead, arsenic. So, any mutation by any means could shape the structure of the DNA anti-bacteria in human body which make human body vulnerable to disease then the entry of so called good bacteria such as food bacteria and yeast would be a foreign agent to our anti-bacteria and it would fight with its immune system which it is late recognition by autoimmune system.

Bacteria come in a variety of size and shapes, with new ones being discovered all the time. Nature has variety in its life forms as you can see just looking at human's guts and whole body. The most common bacterial shapes are Rod, Cocci, and Spiral. However, within each of these groups are hundreds of unique variations.

Summary of experiment: Finding and creating the root cause of Autoimmune Disease in healthy mice as follows: In vitro, the experiment had been done through pure live bacteria of 1 million (CFU) which deformed by microwave heating for less than 5 minutes and injection into mice. In similar procedure, in natural home environment using of any live food bacteria of below (1 through 12) mixed with food products like cheese, are fed by mouth to mice after deforming by microwave for less than 5 minutes. The final results are the same following creation Autoimmune Disease (RA) in mice in vitro and vivo. In lab procedure, more analytical of pure live colony forming units are used for bacteria deformation and denaturation. The following procedures are similar in both mice and human in natural environment that people work, live and unknowingly causing AID in their body over a period of a year or longer, but more concentrated on feed time interval and amount of microbe entry that shows how the infection progress. If you intensify the feed style over time again, you will get AID with these procedures on human or mammalians. It is the fast forward procedure of above experiment that shows the creation of autoimmune disease in mammalian. When the feed time is random and is not consistent, it takes months or year to show the sign of AID, also it is hard to detect it. The slow entries of microbes over period of years are hard to detect from patients, which it makes impossible for researcher to pin point the cause of AID.

The first steps of creating of Autoimmune Disease had been taken by selecting four groups of mice A, B, C and D placebo. In these lab experiments used live food bacteria in size of 1 million colony forming unites (CFU) approximately like a slice cheese of 3-4 OZ with any bacteria such as; L. acidophilus, Lactobacillus Brevis, B. Subtilis, B. Coagulans, then deformation for by microwave or any other deforming agents, then it was heated less than 100 degree temperature F° for 5 minutes or less no longer heat than 5 minutes needed.

In the first group of mice (A) had been given one dosage of a kind of deformed bacteria such as Lactobacillus Brevis, B. subtilis, B. coagulans, a day. The second group of mice (B) is given twice dosages of above kind of deformed bacteria per day and the third group of mice (C) is given three times of dosage of above kind of deformed bacteria daily. Monitoring the root cause of Autoimmune Disease, by the observation of mice, it had shown that mice in Group C that are given three times per day developed quick and severe symptom AID of arthritis joints inflammation in three months and second group mice (B) had shown AID more than four to five months and the first group mice (A) symptom of AID much more slower than other groups over six months. The symptoms of AID mice in groups vary according to dosage and period of test. The more deformed feed bacteria the faster the symptoms of AID appear. As the research shows that the group C after treatment show less mobility than group of A and B, and placebo, also the inflammatory of joints in group C worse than the other A and B group which it is twice or three times more than group A and B.

The second step had taken by treatment of those mice with bacteria root cause of Autoimmune Disease with special probiotics by mouth. The first group of mice (A) improves in less time than other groups (B) and (C) since it had less of the infectious disease. The other groups had taken more time over 6 months to a year with medication to cure. Inflammations decreased by giving probiotics, and then stop given food that contains good bacteria which caused the disease.

A treatment needs to be recognized based on patient status of specific deformed DNA bacteria or yeast DNA infection for specific therapy. After isolating the etiologic agents from the infected area and recognizing specific deformed DNA nucleotide base pairing such as A, C, G, T, and compare it to original copies of microbes and getting the gene sequence of it, then treatment can be done with special selection of probiotics for a long period as long as 2 years and shortest of 6 months. The sequence of DNA structure of anti-bacteria human guts at infected location must be checked for differences in nucleotide base pairing. The deformed DNA structure of specific infectious bacteria in the infected area are changed in few nucleotide base and then applying the close match probiotic that has complete correct nucleotide base pairing which also close resemblance of gut bacteria of healthy people. Then the right procedure for Autoimmune Disease like arthritis must be applied, first specific probiotic DNA that close match to the type of deformed DNA gene. There are medical procedure that other use in this filed is affective like specific antibiotics, and specific bacteriophage could be applied to them if resistance to antibiotic.

Most Autoimmune Diseases are the product of transformation and deformation of DNA of good bacteria and yeasts. DNA of these kinds of bacteria which they are described below is in food products that naturally or artificially made by food processing people, manufacturer, fast food, and frozen that contain good bacteria food too. Also, genetically modified (GM) foods are another example which harmful bacteria are inserted into gene of plant seed; this is not exception to these procedures as mentioned above.

These Good Bacteria are as Follow:

Lactobacillus Sp.; L. acidophilus, L. casei, L. delbrueckii ssp. (bulgaricus) L. cellobiosus, L curvatus, L lactis, L. fermentum, L. plantarum, L. reuteri, L. brevis.

Bifidobacterium Sp.; B. bifidum, B. adolescentis, B. infantis, B. thermophilum, B. longum, B. animalis.

Enterococcus Sp.; Ent. Faecalis, Ent. Faecium.

Streptococcus Sp.; S. cremoris, S. salivarius, S. intermedius, S. diacetylactis.

Bacillus Sp.; B. subtilis, B. licheniformis, B. laterosporus, B. pumilus, B. coagulans, B. clausii, B. polymyxa, B. cereus var toyoi B. polyferminticus.

Pediococcus Sp.; acidilactici, Pediococcus.

Lactic acid bacteria (LAB); however it is in dairy products, Propioni bacterium Shermanii and Bifido bacterium spp. which are not lactic acid bacteria, although Bifido bacterium species do produce lactic acid, are also used in dairy production. In addition, other bacteria such as Camembert, Roquefort and Stilton staphylococci are used in varieties of food products.

Some species of yeasts: Saccharomyces boulardii, S. cerevisiae, Brettanomyces bruxellensis, Candida stellata, Schizosaccharomyces pombe, Torulaspora delbrueckii and Zygosaccharomyces bailii.

The digestive systems of human are filled by billions bacteria of different kinds for digestion of variety of foods which each bacteria is responsible for a type or group of foods. If anything happen to these bacteria either by mutation or destruction by antibiotics then they would not be able to digest certain food as before then the food that carries good bacteria would not be digested and the good bacteria becomes a foreign agent to human body and autoimmune systems want to fight it off. Sometimes, autoimmune system would be successful in fighting off them if the population of autoimmune systems higher than foreign agent and if it is lower than it then it loss the fight and inflammation starts and continue to the later becomes one of kinds of autoimmune diseases.

The following agents change the genomic structure of bacteria genes in human when they become deformed by heating, radiation material such as microwave oven x-Ray, dry freeze, and by acid chemical, indirect or direct sun light, long time exposure to room temperature, strong acidic food with low PH, heavy metals such as mercury, lead, arsenic.

Mutation of Some bacteria that cause disease in mammalian by other researchers: Lactobacillus casei¹ mutation by above deformation agents could cause Autoimmune Diseases, infection in gastrointestinal by Helicobacter. Pylori which Viable L. Casei are required for growth inhibition.

Lactobacillus Paracasei. mutation by above deformation agents could cause Autoimmune Diseases, infection of oral cavity. It has been demonstrated to inhibit the growth of many pathogenic microbes such as Streptococcus mutants, in vitro.²

Lactobacillus plantarum, mutation by above deformation agents could cause Autoimmune Diseases, and infection in abdominal. It is an antibacterial to enteric pathogens like Pseudomonas aeruginosa, Staphylococcus aureus, Escherichia coli, Aeromonas veronii and clinical isolates of P. aeruginosa and E. coli.³

Lactobacillus salivarius, mutation by above deformation agents could cause Autoimmune Diseases. Some strains of S. salivarius are found to produce BLIS (Bacteriocin-like Inhibitory Substances) which is an antimicrobial peptide. This peptide displays interspecies inhibition, and inhibits Streptococcus pyogenes (which causes Strep throat infections).⁴

Lactobacillus Rhamnosus, mutation by above deformation agents could cause Autoimmune Diseases, it is not prevention of rotavirus diarrhea in children,^{5/6/7/8} not reduction in the risk of respiratory tract infections in children, not inhibition or adhesion of vaginal and urinary pathogens, and this bacterium may have an effect on GABA neurotransmitter receptors, which it controls anxiety and stress hormones.⁹

Lactococcus Lactis, mutation by above deformation agents could cause Autoimmune Diseases, stress hormone malfunction.¹⁰

Bacillus Coagulans mutation by above deformation agents could cause Autoimmune Diseases, infection for diarrhea, including infectious types such as rotavirus diarrhea in children; traveler's diarrhea; and diarrhea caused by antibiotics and. Bacillus coagulant is also used for general digestion problems, irritable bowel syndrome (IBS), inflammatory bowel disease (IBD), Crohn's disease, ulcerative colitis), a bowel disorder called Clostridium difficilecolitis, excessive growth of “bad” bacteria in short bowel syndrome, and infection due to the ulcer-causing bacterium Helicobacter pylori.¹¹. Myocarditis caused by deformation of bacteria in dried whey product.

Bifidobacterium longum, mutation by above deformation agents could cause Autoimmune Diseases, obesity and diabetic. “Bifidobacteria have a symbiotic relationship with its human host. B. longum ferments sugars into lactic acid, and this helps to lower the pH in the intestines. Bifidobacteria are known to be probiotic, which means that it's a microbe that helps to protect its host and prevent disease. B. longum is the most often seen species in humans and has been studied to show that it provides potential health benefits. These benefits include: “diarrhea prevention in antibiotic treated patients, cholesterol reduction, alleviation of lactose intolerance symptoms, immune stimulation, and cancer prevention”.¹²

Pediococcus acidilacti, mutation by above deformation agents could cause Autoimmune Diseases, colonization of patagens in the intestine by pathogens like Shigella, Salmonella, Clostridium difficile and Escherichia Coli among small animia¹³
Saccharomyces Boulardii, mutation by above deformation agents could cause Autoimmune Diseases, and Acute diseases; Antibiotic-associated diarrhea, Clostridium Difficile infection, Acute diarrhea, Persistent diarrhea, Enteral nutrition-related diarrhea, Traveler's diarrhea, Helicobacter pylori infection.
Chronic diseases; Crohn's disease, Ulcerative colitis, Irritable bowel syndrome, Parasitic infections; Amebic colitis, Giardiasis, Blastocystis hominis, Human immunodeficiency virus (HIV)-related diarrhea.¹⁴

L. Acidophilus is a natural part of gastrointestinal and vaginal flora and helps to control the growth of Candida Albicans which causes yeast infections¹⁵ if left unopposed, mutation of this bacteria gene DNA of L. acidophilus causes Autoimmune Diseases, yeast infection and digestion problem of food with high in starch.

Lactobacillus Brevis, mutation by the above deformation agents to these bacteria could cause Autoimmune Disease such obesity¹⁶, arthritis, multiple sclerosis, Fibromyalgia syndrome, Crohn's disease.

Lactobacillus Delbrueckii subsp. Bulgaricus, mutation by above deformation agents could cause Autoimmune Diseases, diarrhea and bowel movement disease and inhibition of normal gastric acid secretion has important side effects.¹⁷

For proof, reference and clarification of my research, I bring the historical experiment of using strains of Pneumococcus bacteria, which cause pneumonia in people that was done by Fred Griffith in 1928 and then followed by other researchers like Oswald Avery and his colleagues. My research follows different way of foot step of them in reverse method.

My research proves that one specific good bacteria can be fighting specific bad bacterium by increase population of good bacteria by introducing 10 times population good bacteria to the infectious bacteria or mutant one.

To find cure for specific AID, there is a need to find the specifics sequence of base pairing of DNA of all bacteria of healthy human body, and there is need to develop of techniques for replacing the infected or deformed bacteria gene with good copy of bacteria. Also, there is a need to sequence DNA of whole deformed bacteria in human body of infected person, and to get an estimate of population of each bacterium in healthy people. There is need to see what disease is associated to which human internal bacteria, by deforming of the good bacteria and injecting it to lab mouse and analysis of the action and reaction of health of mouse.

When we have a form of bacteria base disease in any species like human or other mammalian, we should use first population of good bacteria against bad bacteria before using antibiotic. If we increase the population of good bacteria type fighting disease against infectious bacteria at early stage we would get great response of elimination of infectious disease most of times. Oswald Avery and Fred Griffin experiment is reversible. The method is the strains of probiotics treatment that would be effective in arthritis before treatment of antibiotics, since the deformed bacterium DNA and RNA would be transformed into good copy and some would be reshaped by good strains of probiotics. The same way that a bad copy of DNA and RNA of deformed bacterium were transcribed to genome of good bacteria and this would fight with antibody, these DNA and RNA would be reversed to good bacterium by introducing billions of probiotics to human body and some would be replaced by probiotics.

Experimental design and methods of root cause of Autoimmune Disease are used extensively on variety of bacteria with similar infectious property of arthritis with injection to laboratory mice and then testing drug on mice with arthritis cases. While the disease in the early stage of progress improves faster if less than six month and the cure is more achievable.

For Rheumatoid arthritis treatment, it is preferred first removal of infection or killing off the deformed bacteria of injured sites then applying specific probiotics with match DNA of deformed DNA by mouth, then internal muscle injection (IM) of specific antibiotics on location of joints pain.

For all cases of AID, patients must stop take in any dairy product or food that contains live culture, yeast and bacteria, since these materials intensify the population of deformed bacterial in human body which included genetically modified food (GMF) which manufacturer of these kind of food injected bacteria in the seeds of plant in order to kill bugs, and also people who receive blood or donate blood must be sure that blood does not contain AID infection. Healthy individual should receive blood only test negative for AID infection also blood donor. Parents with AID should not give birth to child until complete cure.

The first milestone has been achieved by Creating the root cause of Autoimmune Disease and creating new case of rheumatoid arthritis in mice, the second milestone has achieved by using agents which they are caused the deformed bacteria by deforming agents and the third milestone is done which curing the disease by medicine specific probiotics and prevention of entry of microbes to human body which it had been done on mice and it is shown the proof.

The heredity condition; some numbers of arthritis cases are heredity which does not show symptom quickly in early life span of human until increased population deformed bacteria and then with an accident to joint and injury happen near joints patient feel the joint pain. The significance of matter is to educate and to prevent the healthy population from acquiring the disease and un-healthy population of arthritis to block progression of disease. By education, people would have more knowledge about what is the cause of the disease. In order to keep control of disease, parents with disease should not give birth until they have done tested for disease then cure by gene reconstruction therapy of probiotics, doing so which they eliminate the case of juvenile arthritis.

The goal of my new research is to find the specifics sequence of base pairing of DNA of all bacteria of healthy human body, and there is need to develop of techniques for replacing the infected or deformed bacteria gene with good copy of bacteria. Also, there is a need to sequence DNA of whole deformed bacteria in human body of infected person, and to get an estimate of population of each bacterium in healthy and no healthy people. There is need to see what disease is associated to which good bacteria, by deforming of the good bacteria and injecting it to lab mouse and analysis of the action and reaction of health of mouse.
1) Aliment Pharmacol Ther. 2003 February; 17(3):429-35. Effect of frequent consumption of a Lactobacillus casei-containing milk drink in Helicobacter pylori-colonized subjects. Cats A, Kuipers E J, Bosschaert M A, Pot R G, Vandenbroucke-Grauls C M, Kusters J G. Source Department of Gastroenterology and Hepatology, Academic Hospital Vrije Universiteit, Amsterdam, The Netherlands. a.cats@nki.nl
²⁾ http://www.ncbi.nlm.nih.gov/pmc/articles/PMC3133768/Clin Oral Investig. 2011 August; 15(4): 471-476. Published online 2010 May 26. doi: 10.1007/s00784-010-0423-9
PMCID: PMC3133768 Probiotic Lactobacillus paracasei effect on cariogenic bacterial flora
Li-Chuan Chuang,^{1,2} Chiung-Shing Huang,^{2,3} Li-Wei Ou-Yang,¹ and Shiao-Yu Lin¹
3) BMC Microbiol. 2011 Jun. 27; 11:152. doi: 10.1186/1471-2180-11-152. Lactobacillus plantarum (VR1) isolated from an ayurvedic medicine (Kutajarista) ameliorates in vitro cellular damage caused by Aeromonas veronii.
Kumar H, Rangrez A Y, Dayananda K M, Atre A N, Patole M S, Shouche Y S. Source Molecular Biology Unit, National Centre for Cell Science, Ganeshkhind, Pune, Maharashtra, India.
4) Wescombe, P. A., N. C. K. Heng, et al. (2009). “Streptococcal bacteriocins and the case for Streptococcus salivarius as model oral probiotics.” Future Microbiology 4(7): 819-835.
5) Canaani, R B; Cirillo P, Terrin G, Cesarano L, Spagnuolo M I, De Vincenzo A, Albano F, Passariello A, De Marco G, Manguso F, Guarino A (2007). “Probiotics for treatment of accute diarrhoea in children: randomised clinical trial of five different preparations”. BMJ 335 (7615): 340. doi:10.1136/bmj.39272.581736.55. PMC 1949444. PMID 17690340. Retrieved 2009-12-03.
6) Österlund, P; Ruotsalainen T, Korpela R, Saxelin M, Ollus A, Valta P, Kouri M, Elomaa I, Joensuu H (2007). “Lactobacillus supplementation for diarrhoea related to chemotherapy of colorectal cancer: a randomised study”. Br. J. Cancer 97 (8): 1028-34. doi:10.1038/sj.bjc.6603990. PMC 2360429. PMID 17895895. Retrieved 2009-12-03.
7) Guandalini, S; Pensabene L, Zikri M A, Dias J A, Casali L G, Hoekstra H, Kolacek S, Massar K, Micetic-Turk D, Papadopoulou A, de Sousa J S, Sandhu B, Szajewska H, Weizman Z (2000). “Lactobacillus G G administered in oral rehydration solution to children with acute diarrhea: a multicenter European trial”. J Pediatr Gastroenterol Nutr 30 (1): 54-60. doi:10.1097/00005176-200001000-00018. PMID 10630440.
8) Vanderhoof, Jon A.; et al. (1999). “LactobacillusGG in the prevention of antibiotic-associated diarrhea in children”. The Journal of Pediatrics 135 (5): 564-568. doi:10.1016/S0022-3476(99)70053-3. Retrieved 2012-04-15.
9) Science News, Belly bacteria boss the brain, Aug. 29, 2011
10) Antonie Van Leeuwenhoek. 1996 October; 70(2-4):243-51. Lactococcus lactis and stress. Rallu F, Gruss A, Maguin E. Source Institut National de la Recherche Agronomique Laboratoire de Génétique Microbienne, France.
11) Czaczyk K, Tojanowska K, Mueller A. Antifungal activity of Bacillus coagulans against Fusarium sp. Acta Microbiol Pol 2002; 51:275-83.
Donskey C J, Hoyen C K, Das S M, et al. Effect of oral Bacillus coagulans administration on the density of vancomycin-resistant enterococci in the stool of colonized mice. Lett Appl Microbiol 2001; 33:84-8.
Duc L H, Hong H A, Barbosa T M, et al. Characterization of Bacillus probiotics available for human use. Appl Environ Microbiol 2004; 70:2161-71.
Hyronimus B, Le Marrec C, Urdaci M C. Coagulin, a bacteriocin-like inhibitory subtances produced by Bacillus coagulans 14. J Appi Microbiol 1998; 85:42-50.
McGroarty J A. Probiotic use of lactobacilli in the human female urogenital tract. FEMS Immunol Med Microbiol 1993; 6:251-64.
Probiotics for antibiotic-associated diarrhea. Pharmacist's Letter/Prescriber's Letter 2000; 16(1):160103.
Reid G, Bruce A W, Cook R L, et al. Effect on urogenital flora of antibiotic therapy for urinary tract infection. Scand J Infect Dis 1990; 22:43-7.
Velraeds M M, van der Mei H C, Reid G, Busscher H J. Inhibition of initial adhesion of uropathogenic Enterococcus faecalis by biosurfactants from Lactobacillus isolates. Appl Environ Microbiol 1996; 62:1958-63.
12) Doe Joint Genome Institute: Bifidobacterium longum. 2004
13) Recent Understandings and Biomedical Applications Jamila K Adam a, BhartiOdhav b and K Suresh Babu Naidua* a Department of Biomedical and Clinical Technology, Durban University of Technology, Durban-4000, South Africa Department of Biotechnology and Food Technology, Durban University of Technology, Durban-4000, South Africa
14) Therap Adv Gastroenterol. 2012 March; 5(2): 111-125. doi: 10.1177/1756283X11428502
American Physiological Society
American Society for Microbiology
American Society for Nutrition
CBE-Life Sciences Education (American Society for Cell Biology)
Genome Research (Cold Spring Harbor Laboratory Press)
Journal of Cell Biology (Rockefeller University Press)
Journal of Experimental Medicine (Rockefeller University Press)
Journal of General Physiology (Rockefeller University Press)
Molecular Biology of the Cell (American Society for Cell Biology)
National Academy of Sciences
Nucleic Acids Research (Open Access content)
Physical Therapy
15) Yuan-Kun Lee [ ] (2009). Rev. ed. of: Handbook of probiotics 2nd ed. Hoboken, N.J.: John Wiley & Sons. pp. 441-443.
16) U.S. Pat. No. 8,497,114 Kondo et al. Jul. 30, 2013
Kondo; Shizuki Yokohama from Japan. Shimizu; Kanetada Zama
17) J Clin Gastroenterol. 2012 October; 46 Suppl:S18-26. doi: 10.1097/MCG.0b013e318267b55d. Del Piano M, Anderloni A, Balzarini M, Ballarè M, Carmagnola S, Montino F, Orsello M, Pagliarulo M, Tari R, Soattińi L, Sforza F, Mogna L, Mogna G Department of astroenterology, Gastroenterology Unit, Maggiore della Inventor: Research Scientist Majid Kazemi

1) I claim that cause of autoimmune diseases are good bacteria that turned to, deformed, or transformed by chemical, and environmental agents to bad bacteria.

2) DNA structure of good bacteria is mutated, deformed by chemical environmental agents.

3) Why Autoimmune Disease is not recognized by autoimmune systems.

4) Mutated, Deformed DNA should be removed and replace with the same human bacteria.

5) Nucleotide changes of deformed DNA happen at nucleotide base pairing such as A, C, G, T, and U.

6) The following agents change the genomic structure of good bacteria genes in human in which the root cause of 172 autoimmune disease when they become mutated, deformed by heating, radiation material such as; microwave oven, x-Ray, and dry freeze, acid chemical, indirect sun light, longtime exposure to room temperature, strong acidic food with low PH, heavy metals such as mercury, lead, arsenic.
